(19) United States
(12) Patent Application Publication
(10) Pub. No.: US 2026/0158211 A1
(43) Pub. Date: Jun. 11, 2026

(54) PERSONALIZED CHRONOTHERAPY

(71) Applicant: CareFusion 303, Inc., San Diego, CA (US)

(72) Inventor: Michael Anthony BOYLAN, Athboy (IE)

(21) Appl. No.: 18/977,466

(22) Filed: Dec. 11, 2024

(51) Int. Cl. A61M 5/142; G16H 20/17; G16H 40/63

(52) U.S. Cl. CPC A61M 5/142; G16H 20/17; G16H 40/63; A61M 2005/14208; A61M 2005/14292; A61M 2202/0007; A61M 2205/3303; A61M 2205/3327; A61M 2205/50; A61M 2230/005

(57) ABSTRACT

Certain aspects of the disclosure provide techniques for personalized chronotherapy. An example method includes obtaining sensor data associated with one or more sensors; determining a circadian rhythm based on the sensor data; determining a control schedule, for a device configured to deliver a therapeutic, based on the circadian rhythm and pharmacological data associated with the therapeutic; and controlling the device in accordance with the control schedule.

### BACKGROUND

### Field

Aspects of the present disclosure relate to device control for personalized chronotherapy, for example, determination of and control of a device for treatment in accordance with a personalized chronotherapy schedule.

### Description of Related Art

A circadian rhythm is the approximately twenty-four hour cycle of biological oscillations. All living things, including humans, have a circadian rhythm. In humans, the circadian rhythm is indicative of wake and sleep periods, rest and activity periods, appetite, muscular strength and performance due to biochemical and hormonal changes. The circadian rhythm may exist without environmental influence, however, environmental cycles and cues may affect the circadian rhythm pattern. For example, regular alternation of light and darkness, physical activity and rest, social interactions, feeding patterns, and/or the like may alter the circadian rhythm.

The superchiasmatic nuclei (SNC) is a bilateral structure located in the anterior part of the hypothalamus. It is the central pacemaker of the circadian timing system and regulates most circadian rhythms in the human body. The SCN integrates light and dark information from the retinal ganglion cells in the eye and generate rhythmic signals affecting neuromediator tracking, body temperature, cytokines, and hormonal secretions. SCN-dependent rhythmic signals work to coordinate genetic molecular clocks that reside within each cell, resulting in a hierarchical clocks network that form the circadian rhythm. For example, within each cell transcription-translation feedback loops control mRNA expression and other gene expression patterns.

At a tissue level, the circadian rhythm controls metabolism and proliferation of organs and tissues, for example, liver, heart, lung, kidney, intestine, pancreases, muscle, breast, brown fat, and skin over the 24 hour cycle. Further, the molecular circadian rhythm is involved in stress response and cell cycle control. Specifically, the molecular circadian rhythm is involved with the process of verifying whether processes at each phase of a cell cycle have accurately completed before progressing to the next phase.

Several genes, including BMAL1/BMAL2, CLOCK, CRY1/CRY2, and PER1/PER2/PER3, play a crucial role in regulating transcription and translation. The expression of these core clock genes within the cell influences various signaling pathways, enabling cells to recognize the time of day and carry out their appropriate functions. Additionally, the phosphorylation of core clock proteins results in their degradation, helping to maintain synchronization within the 24-hour cycle.

Furthermore, mutations in various genes involved in sleep are associated with alterations in circadian rhythm. For example, Period (e.g., PER2) is associated with advanced sleep phase syndrome. Casein kinase mutations CSNK1D, or CSNK1E are associated with advanced sleep phase syndrome and delayed sleep phase syndrome respectively. Circadian Locomotor Output Cycles Kaput (CLOCK) variant is associated with diurnal sleep.

Each person's circadian rhythm may be unique to them. Further, in some cases, a circadian rhythm may shift. For example, as a person ages their sleep patterns may shift, especially towards earlier sleep and wake periods, activity levels may decline, and daytime sleepiness may increase. Sleep quality can also diminish. Additionally, sensitivity to external cues, such as light, may decrease as a person ages, which may shift or otherwise disrupt their circadian rhythm. Infants generally develop their circadian rhythm around 3 to 6 months and patterns of sleep may develop. As another example, some health conditions, such as disease, illness, or medication may impact a person's circadian rhythm. Specifically, cancers, metabolic, neurodegenerative, psychological, and cardiovascular diseases may be associated with shifts or disorder of a person's circadian rhythm.

### SUMMARY

Certain aspects provide a personalized chronotherapy method. The method includes obtaining sensor data associated with one or more sensors; determining a circadian rhythm based on the sensor data; determining a control schedule, for a device configured to deliver a therapeutic, based on the circadian rhythm and pharmacological data associated with the therapeutic; and controlling the device in accordance with the control schedule.

Certain aspects provide an infusion pump controller comprising: one or more memories; and one or more processors, coupled to the one or more memories, configured to: obtain sensor data associated with one or more sensors for a patient; determine a circadian rhythm based on the sensor data; determine a control schedule for an infusion pump configured to deliver a therapeutic to the patient, based on the circadian rhythm and pharmacological data associated with the therapeutic; and cause the infusion pump to be configured for the patient in accordance with the control schedule.

Other aspects provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

The following description and the related drawings set forth in detail certain illustrative features of one or more aspects.

### DESCRIPTION OF THE DRAWINGS

The appended figures depict certain aspects and are therefore not to be considered limiting of the scope of this disclosure.

FIG. 1 depicts an example personalized chronotherapy system.

FIG. 2 depicts an example workflow for controlling a therapeutic system configured to administer personalized chronotherapy to a subject.

FIG. 3 depicts an example personalized circadian rhythm for a subject.

FIG. 4 depicts an example personalized chronotherapeutic schedule for a subject.

FIG. 5 depicts an example infusion pump, for example, configured to administer therapy to a subject, in accordance with a personalized chronotherapeutic schedule.

FIG. 6 depicts an example syringe pump, for example, configured to administer therapy to a subject, in accordance with a personalized chronotherapeutic schedule.

FIG. 7 depicts an example method for controlling a therapeutic system configured to administer personalized chronotherapy to a subject.

FIG. 8 depicts an example processing system with which aspects of the present disclosure can be performed.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for administering personalized chronotherapy, for example, for controlling one or more devices (e.g., a system) configured to deliver a therapeutic (e.g., infusion-based therapy), such as to a subject, based on a circadian rhythm (e.g., personal circadian rhythm of a subject) and therapy prescription. Certain aspects may generally refer to administration of personalized chronotherapy to a subject. However, it should be understood that such administration of personalized chronotherapy may refer to controlling one or more devices based on a circadian rhythm, and may not require providing actual treatment on a subject.

Two biomarkers of circadian rhythms are the hormones melatonin and cortisol. Melatonin is a hormone produced by the penal gland in the brain during darkness. For example, melatonin may begin to rise from around 18:00-22:00, peaking at around 02:00-04:00, before falling towards a baseline by 10:00, which persists throughout the day. In the absence of light, production of melatonin by the pineal gland is not suppressed and melatonin level rises. Visual light entering the eye triggers suppression of melatonin by the pineal gland through SCN-dependent pathways. Peak melatonin levels may be different in individuals, for example, based on endogenous timing, routines, light exposure and/or the like. For example, light exposure for several consecutive nights may shift peak melatonin concentration, for example, shifting the peak later (e.g., early the next morning).

Melatonin functions as an antioxidant in addition to serving as a chemical expression of darkness. Melatonin is a derivative of the amino acid, tryptophan. Besides production in the penal gland, regulated by light, other sources of melatonin include microbiota production (e.g., within the mammalian microbiome of skin, digestive track, and orifices), mitochondria production, and consumption through diet. Melatonin deficiency is associated with a variety of disorders including neurodegenerative diseases, heart diseases, metabolic disorders, osteoporosis, cancer, and even aging.

Cortisol is a steroid hormone produced by the adrenal gland under control of the hypothalamic-pituitary-adrenal axis. Cortisol is released in response to stress and low blood glucose. Cortisol functions to increase blood glucose through gluconeogenesis, suppress the immune system, and otherwise aid in metabolism. Cortisol is moderated by stress and may impact inflammation, depression, post-traumatic stress disorder, cardiovascular disease, type II diabetes, or stroke.

Cortisol levels may peak in the early morning, around 08:00, before falling during the day to a baseline at around 00:00-02:00, when melatonin peaks. Cortisol levels may start rising around 04:00, when melatonin begins to decline.

Body temperature also follows the circadian rhythm and fluctuates through a 24 hour period, typically higher during the daytime and lower during the nighttime. In addition, body temperature fluctuates during certain sleep cycles, including dropping during non-REM sleep. Body temperature then rises before waking. For example, central body temperature is tightly regulated in mammals so it usually varies from <36.5 *C near 02.00 at night to >37.5 *C around 18:00 in the late afternoon. Additionally, melatonin also reinforces decrease of central temperature in the evening, before sleep, as a result of vasodilation-associated heat loss that occurs at the extremities and at skin surface level and can be influenced by physical activity. This decrease in body temperature may trigger sleep onset.

Similarly, rest and activity vary with the circadian rhythm. For example, rest patterns may indicate both nighttime sleep, as well as mid-day sleep such as naps. Additionally, activity patterns may indicate exercise, including peak performance, as well as general increased alertness. For example, generally peak alertness and performance occur during the later morning and early afternoon, e.g., 10:00-14:00. However, there is often a dip in alertness in the early afternoon. Alertness may also rise in a moderate peak in the evening before decreasing around bedtime. Alertness may be tied to both cognitive and physical performance.

Circadian rhythms may shift, or otherwise be disrupted or altered. For example, each individual's circadian rhythm may be personalized, based on their genetics and environment. So-called “early birds” and “night owls” may have shifted circadian rhythms, early birds shifted earlier than night owls. Further, environmental factors, such as later or earlier light exposure, later or earlier activity, etc. may also shift an individual's circadian rhythm. For example, night-time shift workers may have a later circadian rhythm due to their work schedule.

Other lifestyle factors may similarly affect an individual's circadian rhythm. Generally, an individual's circadian rhythm starts forming patterns by about 3-6 months, and then stabilize during adolescence and early adulthood. The individual's circadian rhythm may generally remain the same, absent other factors, until advanced age, when it may shift earlier.

Furthermore, some health issues may be associated with disrupted or altered circadian rhythms. In some cases, a health issue may cause the altered circadian rhythm. For example, cancer may cause disruptions to a circadian rhythm. In one example, an altered pattern of cortisol secretion, such as flattened or reversed diurnal variation may be associated with poor survival in patients with metastatic or advanced breast, ovarian, kidney or lung cancer. As another example, patients with insomnia, depression or schizophrenia may have low melatonin secretion and dosing additional melatonin in the evening may improve sleep quality, blood pressure, metabolism, and mood, and help prevent earlier morning cortisol peak. In yet another example, hypercholesterolemia is associated with increased hepatic cholesterol synthesis in the evening and night. For peptic ulcer disease, there is an increase in gastric secretion in the evening and night. Pain often increases between 3:00 and 8:00. Migraine episodes often occur between 8:00 and 10:00 with prodromal disturbances in early morning hours. Endocrine disorders, including Addison's disease results in a complete deficiency of adrenal steroids and replacements are administered to coincide with the circadian variability of the hypothalamic-pituitary-adrenal axis. For subjects with diabetes, blood glucose levels and insulin levels change with the circadian rhythm, often hyperglycemia overnight between 4:00 and 8:00.

In other cases, an altered circadian rhythm may be correlated with a health issue. For example, there is evidence that prolonged nighttime shift work is associated with an increased risk of developing cancer. Melatonin suppression or circadian disruption may be causes.

Moreover, in some cases, it may be a treatment for a health issue, instead of or in conjunction with the health issue that is associated with the altered circadian rhythm. For example, fatigue is characterized by a sense of weariness that affects physical and/or mental capabilities, making it difficult to engage in physical or intellectual tasks. In healthy individuals, feeling fatigued is normal after a day of work or following physical or mental exertion. While this kind of fatigue can occasionally be bothersome, it typically has minimal impact on daily life and can be alleviated by a good night's sleep. For cancer patients, however, both the disease and treatment can induce significant fatigue, even 5 years after cancer treatment. While the causes are not known, disruption to rest-activity rhythm, flattened diurnal cortisol rhythms, and/or pro-inflammatory cytokines could play a role.

Pharmaceuticals, such as those used in cancer treatment, may be associated with altered circadian rhythm due to their pharmacokinetic and pharmacodynamic activity. Pharmacokinetics characterizes how a pharmaceutical is absorbed, distributed, metabolized, and excreted in the body. Absorption characterizes how the pharmaceutical enters the bloodstream after administration, for example, some pharmaceuticals may be ingested orally, intravenously, intramuscularly, subcutaneously, rectally, inhalation, etc. Distribution characterizes how the pharmaceutical spreads throughout the body to tissue and organs. The efficacy or toxicity of a drug depends on the distribution in specific tissues and in part explains the lack of correlation between plasma levels and the effects that are seen. Based on the molecular structure, drugs have variable distribution in different types of tissues such as fat, muscle, and brain. Unlike other tissues, the brain and testes are unique, as they contain membrane barriers making a drug significantly less susceptible to distribution. Metabolism is the process by which the body chemically breaks down to create compounds for excretion from the body, often in the liver. Drug elimination is the sum of the processes of removing an administered drug from the body.

Pharmacodynamics is characterizes the effects of drugs on the body and the mechanisms through which they exert their effects, including the mechanism of action, drug-receptor interactions, dose-response relationship, therapeutic and adverse effects. Mechanism of action characterizes how a drug produces its effects at the molecular, cellular, or systemic level, and may include interactions with specific receptors, enzymes, or biological pathways. Drug-receptor interactions characterize the binding of a drug to its target receptors, which can lead to changes in cellular activity and physiological responses. Drug-response relationship is the relationship between the dose of a drug and its magnitude of its effects, which helps to determine the optimal dosage for therapeutic efficacy. Therapeutic effects are the desired effects of the drug, including efficacy in treating a particular condition. Adverse effects are unintended or harmful effects that may occur as a result of a drug action.

The pharmacokinetics and pharmacodynamics of a drug may influence its temporal dosage. Indeed, diurnal oscillations in drug absorption, distribution, metabolism, and excretion (ADME), as well as daily variations in the sensitivity of molecular drug targets leads to the dosing-time dependencies. For example, some drugs are prescribed at regular intervals (e.g., 8, 12, 24 hours) to maintain effective blood levels. Some drugs are taking at specific time(s) to mimic natural rhythm, reduce adverse effects, or optimize absorption. These include hormones taken at the time of natural rise, such as corticosteroids in the morning to coincide with increasing cortisol levels, selective serotonin reuptake inhibitors (SSRIs) in the morning to reduce insomnia and increase focus and motivation, statins at night to coincide with increased cholesterol production around midnight, and/or the like. This practice of timing administration of a treatment or therapy to align with the body's circadian rhythm is called chronotherapy.

Generally, current chronotherapy practices involve a patient's physician and/or pharmacist considering the patient's individual physiological and pathophysiological conditions, for example, age, sex, activity, symptoms, comorbidities, current prescriptions, medical history, and/or the like, as well as the pharmacology of the therapeutic itself, including clinical trials and clinical practice guidelines to determine a dosing recommendation.

However, as described herein above, each individual has an individual and personalized circadian rhythm. Generally, dosing times for chronotherapy are based on research studies, such as an average or typical circadian rhythm, and not the individual's circadian rhythm. Thus, the dosing may not be personalized. Furthermore, an individual's circadian rhythm may change, e.g., due to age, changes in lifestyle or environmental factors, or health issues. As such, dosing times may need to be changed over time as an individual's circadian rhythm changes.

Aspects of the present disclosure provide for systems and methods for chronotherapy, for example, controlling one or more devices configured to deliver a therapeutic based on a circadian rhythm and therapy prescription. For example, in some aspects, an infusion system may be configured to administer a therapy to a subject based on a circadian rhythm. In certain aspects, the circadian rhythm may be specific to the subject, and administration of the therapy is based on the subject's personalized circadian rhythm. In particular, aspects of the present disclosure provide for utilizing sensor data, including sensor data associated with a circadian rhythm, such as cortisol and melatonin, temperature, activity, heart rate, and/or the like, to determine a circadian rhythm. For example, in certain aspects, a machine learning model may be used to process such sensor data and determine the circadian rhythm. In certain aspects, the sensor data associated with a subject may be used to determine the subject's personalized circadian rhythm, for example, using a machine learning model.

Based on the pharmacokinetics and pharmacodynamics of the therapy(ies), a therapeutic schedule may be determined in accordance with and enhanced by, the circadian rhythm. The therapeutic schedule may also be referred to as a control schedule, and, in some aspects, may include instructions for controlling a device configured to deliver a therapeutic. For example, in certain aspects, a drug associated with an increase in cortisol may be beneficially scheduled for administration in the morning to coincide with a natural increase in cortisol in the morning. In one example, a subject with a predicted cortisol peak around 8:00 may beneficially be scheduled to be treated with the drug at or just before 8:00 to coincide with the subject's personal predicted peak of 8:00.

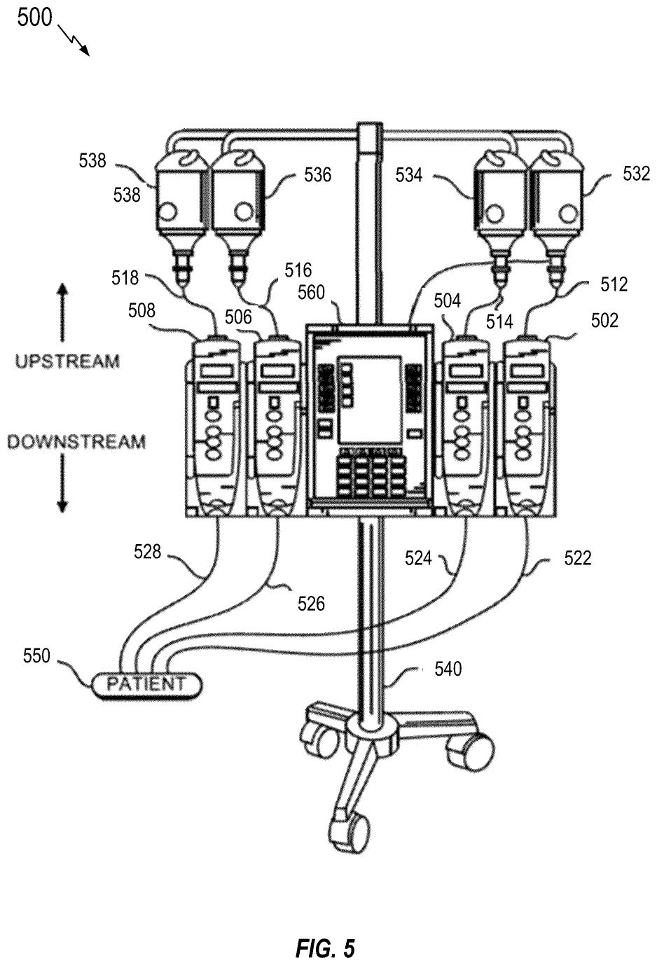

Then, in certain aspects, a therapeutic may be administered in accordance to the therapeutic schedule. In some aspects, a device configured to deliver a therapeutic may be controlled based on a control schedule to administer the therapeutic. For example, in some aspects, an infusion pump, such as infusion system 500 in FIG. 5, may be configured to control administration of the therapeutic according to the therapeutic schedule. As another example, a dialysis machine may be configured to control administration of the therapeutic according to the therapeutic schedule.

Beneficially, the therapy may have improved efficacy, for example, by coinciding with one or more circadian-dependent biological functions. Further, adverse effects may be reduced by coinciding the pharmacological effects of the therapy with one or more circadian-dependent biological functions. For example, by coinciding with one or more circadian-dependent biological functions a reduction in disruption or restoration of robust circadian function may be achieved. Further, beneficial treatment effects and adverse treatment effects may be optimized. Additionally, in some aspects, other activities, such as meal consumption, exercise, sleep, and/or the like may also be timed with the therapy to improve restoration or enhancement of circadian rhythm and avoid disruption. Thereby, improved treatment and survival outcomes, as well as improved patient quality of life may be achieved.

### Example Personalized Chronotherapy System

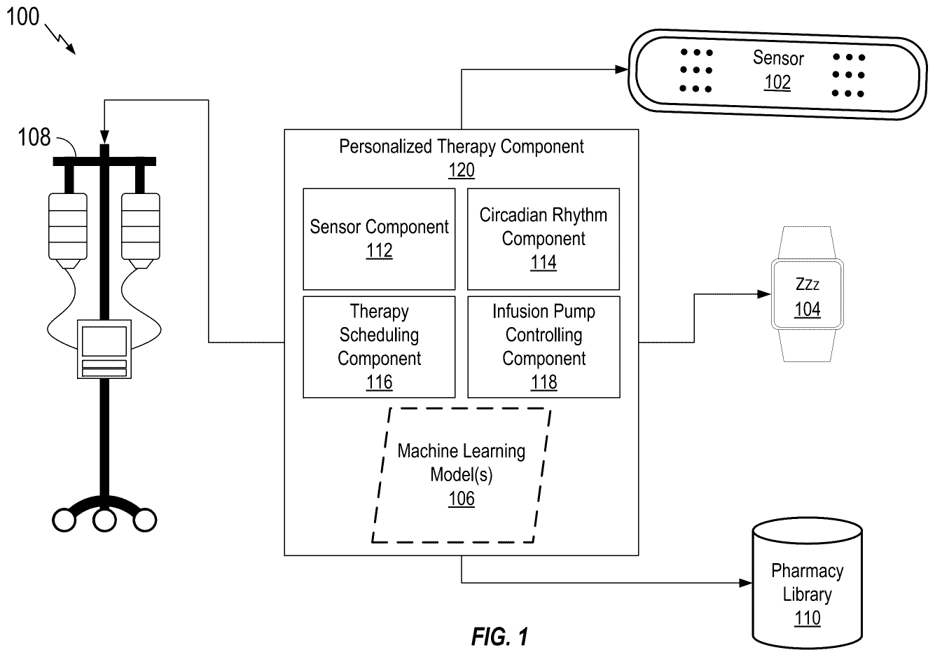

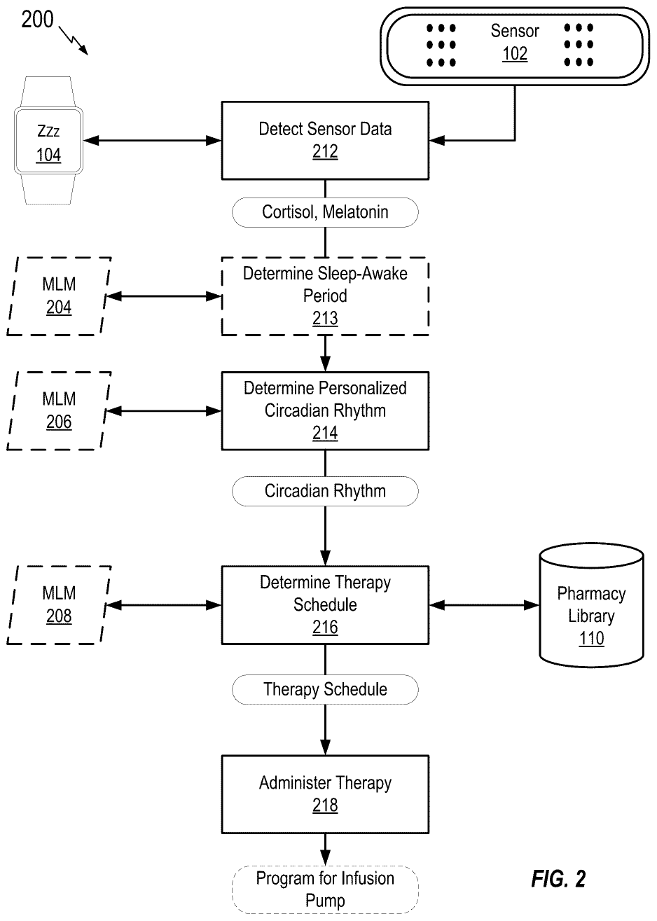

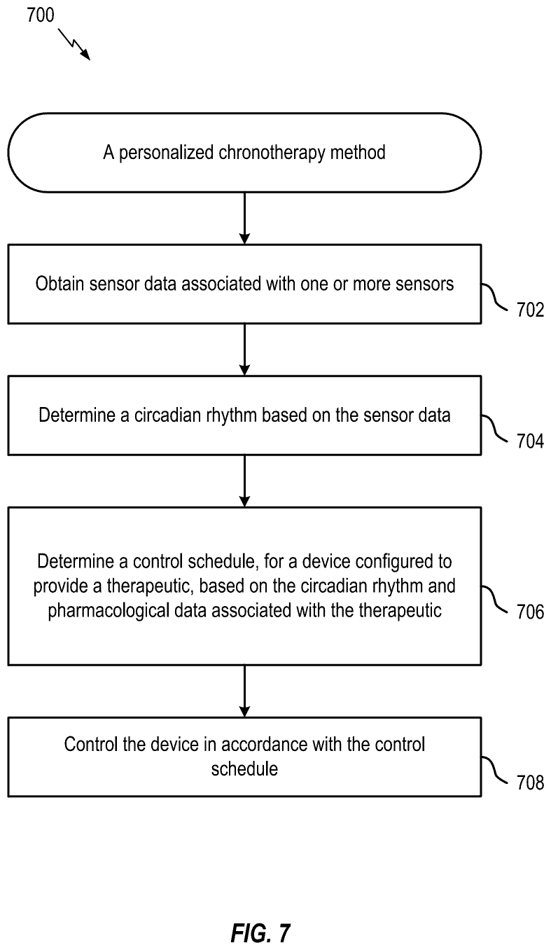

FIG. 1 depicts an example personalized chronotherapy system 100 configured to control personalized chronotherapy, such as for a subject. Personalized chronotherapy system 100 is configured to implement systems and methods, for example, workflow 200 in FIG. 2 and method 700 in FIG. 7, to control personalized chronotherapy.

Personalized chronotherapy component 120 is configured to determine a circadian rhythm, in an illustrative example, a subject's personalized circadian rhythm, and determine a control schedule (also referred to as a therapeutic schedule) based on the circadian rhythm and the pharmacokinetics and pharmacodynamics of the therapeutic. Then, in certain aspects, the therapeutic may be administered in accordance to the personalized therapeutic schedule, for example, by controlling a medical device configured to administer the therapeutic.

In this example, personalized chronotherapy component 120 comprises a sensor component 112, a circadian rhythm component 114, a therapy scheduling component 116, an infusion pump controlling component 118, and one or more machine learning models 106. A machine learning model may receive a set of inputs including sensor readings, patient information (e.g., age, weight, gender, etc.) and provide, as one or more output values, a predicted circadian rhythm. The model may be trained using a set of known inputs and verified circadian rhythms.

Sensor component 112 is configured to obtain and process sensor data captured by a sensor 102 and/or a smart device 104. In some aspects, multiple sensors may be utilized. The sensor 102 and/or the smart device 104 are configured to sense one or more biomarkers, such as associated with a subject. A biomarker is a measurable indicator of a biological condition or process. It can be a substance, such as a protein or gene, found in blood or tissues that reflects normal or abnormal biological activity or a disease state. For example, a biomarker may include one or more analytes. An analyte is a substance or component which may be measured or analyzed, such as cortisol, melatonin, growth hormone (GH), testosterone, insulin, glucose, thyroid-stimulating hormone (TSH), triiodothyronine (T3), thyroxine (T4), epinephrine, nitric oxide, acetone, C-reactive protein,, Hormone ER or Hormone ER2, other hormones, other proteins, other electrolytes, other phospholipids, and/or the like. In another example, a biomarker may include one or more characteristics, such as of the subject, for example, activity rate of the subject, a heart rate (HR), a heart rate reserve, a heart rate variability (HRV), respiration rate, electrocardiogram (e.g., ECG or EKG) data, temperature, impedance data, step count, or the like.

In some aspects, a sensor is a chemical or biochemical sensor, for example, an analyte sensor. In some aspects, a sensor may be a multi-analyte sensor. In some aspects, a sensor is an optical sensor or an electrical sensor. In some aspects, a sensor is an accelerometer, gyroscope, global positioning system (GPS), or a barometer. In some aspects, a sensor is a blood oxygen sensor, a heart rate monitor, a thermometer, a breath analyzer, or the like.

In the depicted example, sensor 102 may comprise a skin-patch sensor, or other wearable sensor. For example, sensor 102 may comprise a sweat sensor configured to measure one or more biomarker present in sweat, and/or a skin temperature. Biomarkers measurable by a sweat sensor may include sodium, potassium, chloride, glucose, lactose, urea, ammonia, short-chain fatty acids, cortisol, creatinine, melatonin, testosterone, estrogen, temperature, and/or the like. In one example, sensor 102 may comprise a subcutaneous sensor configured to measure one or more biomarkers present in interstitial fluid, for example, glucose, potassium, calcium, magnesium, enzymes, hormones, lactate, urea, creatinine, fatty acids and lipid compounds, cellular debris, temperature, or the like.

In some aspects, sensor 102 may be configured to sense one or more biomarkers in blood, saliva, or urine of a subject. For example, biomarkers in blood may include electrolytes, enzymes, hormones, proteins, lipids, metabolites, blood cells, and/or the like. Biomarkers in saliva may include cortisol, testosterone, estradiol, enzymes such as amylase, lactate dehydrogenase, electrolytes such as sodium, potassium, and calcium levels, other proteins, glucose, uric acid, and lactate, viral RNA, microbial DNA, and/or the like. Biomarkers in urine may include electrolytes such as sodium, potassium, calcium, and magnesium, proteins such as albumin, creatinine, amino acids (e.g., ketones), immunological proteins (e.g., immunoglobulins) or cells (e.g., cytokines), glucose, urea, creatinine, uric acid, hormones such as cortisol, epinephrine, progesterone, and/or the like.

In some aspects, sensor 102 may be configured to measure one or more environmental conditions associated with the subject, for example, environmental temperature, environmental light conditions, environmental noise conditions, etc.

Smart device 104 may be a smart device, for example, a smart watch, smart ring, smart jewelry, mobile device, fitness or activity monitor, and, the like. In some examples, sensor 102 may be incorporated or coupled to the smart device 104. In some aspects, sensor 102 and/or smart device 104 may be configured to continuously monitor or periodically monitor biomarkers, such as of the subject, over time. In some examples, sensor 102 and/or smart device 104 may be utilized for a period of time, such as a 24 hour period, a weekly period, etc. In some examples, sensor 102 and/or smart device 104 may be utilized continuously, such as monitoring of biomarkers continuously, continually, and or intermittently (regularly or irregularly), for example, about every 5 to 10 minutes.

In some cases, sensor component 112 is configured to receive additional data associated with a subject, for example, age, sex, exercise or activity data, nutrition data, sleep data, work schedule, and/or the like. In some aspects, sensor component 112 is configured to process sensor data and determine patterns, for example, an elevated HR may indicate a user is active. In some cases, sensor component 112 may correlate data from multiple sensors (or multiple types of data), for example, an elevated HR and accelerometer data may indicate a user is active.

Sensor component 112 is further configured to time-stamp sensor data, including, for example, time of day, day of week, week of year, etc.

The circadian rhythm component 114 is configured to determine a (e.g., subject's personalized) circadian rhythm, for example, as described with respect to workflow 200 in FIG. 2. A personalized circadian rhythm comprises a subject's internal regulation of biological processes, including sleep-wake cycle, hormone regulation, metabolism, body temperature cycle, blood pressure cycle, etc., specific to that subject. As described herein, in some cases, a subject's circadian rhythm may shift, alter, or otherwise be perturbed such that a personalized circadian rhythm may alter over time.

In some aspects, a subject's personalized circadian rhythm is characterized by the timing of the subject's physiological functions, such as sleep-wake cycle, hormone levels, metabolic processes, mental acuity, and/or the like. In some aspects, a subject's personalized circadian rhythm is characterized by the timing of the subject's activity, for example, diet (e.g., consumption of food), exercise, physical or mental activity (e.g., work), and/or the like. In some aspects, a subject's personalized circadian rhythm is characterized by the timing of the subject's environment, such as daylight (e.g., sunrise and sunset), season (e.g., summer or winter), time zone, and/or the like.

In some aspects, a subject's personalized circadian rhythm is defined by a time of the event (or expected time of the event). For example, a cortisol peak at 6:00, a sleep cycle beginning at 20:00, or a melatonin peak at 2:00.

In some aspects, a subject's personalized circadian rhythm is defined by one or more periods, during which one or more events (or expected events) may occur. For example, a personalized circadian rhythm may comprise four periods. A first period may be defined from when the subject wakes to the middle of a wake period (e.g., a morning period). A second period may be defined from the middle of the wake period to beginning of a wind-down period (e.g., an afternoon period). A third period may be defined from the beginning of the wind-down period to when the subject sleeps (e.g., an evening period). A fourth period may be defined from when the subject sleeps to when the subject wakes (e.g., a night period).

In some cases, such as for shift workers, a subject's personalized circadian rhythm is such that timing of one or more events may not correspond with a typical timing. For example, a first period may begin at 18:00 and end at 0:00, when the subject wakes up and begins their day. Thus, in some aspects, a subject's personalized circadian rhythm may be defined based on the event(s) and/or the period, for example, a cortisol peak is expected 2 hours before the subject wakes up, rather than a specific time (e.g., 6:00).

In some cases, timing of a peak level of a biomarker may be referred to as the acrophase of the biomarker. The average level of the biomarker over a 24 hour period may be referred to as the MESOR. The timing of switching between low and high activity of the biomarker may be referred to as the up-MESOR, while switching between high and low activity of the biomarker may be referred to as down-MESOR. The difference between the maximum and minimum level of the biomarker may be the amplitude of the biomarker, while the difference between the maximum and minimum levels of the cosine function may be the double amplitude of the biomarker.

In some aspects, the circadian rhythm is based on the sensor data obtained and processed by sensor component 112.

In some aspects, a subject's personalized circadian rhythm may be determined in a deterministic matter, for example, based on an biomarker satisfying a threshold, e.g., a maximum or minimum threshold, or by comparison to a reference value. For example, a cortisol value above a maximum threshold at 8:00 may be considered a morning peak.

In some aspects, the circadian rhythm component 114 is configured to utilize one or more machine learning models 106 to determine a circadian rhythm.

In some aspects, the circadian rhythm component 114 is further configured to utilize additional data associated with the subject, for example, a subject's medical history, one or more comorbidities, age, sex, current therapeutics, and/or the like. In some aspects, the circadian rhythm component 114 is further configured to utilize environmental data associated with the subject, for example, the environmental conditions associated with the subject, such as environmental temperature, environmental light conditions, and environmental noise conditions.

In some aspects the circadian rhythm component 114 is further configured to utilize sensor data, additional data, and/or environmental data associated with the subject to determine a sleep-wake cycle of the subject. A sleep-wake cycle may comprise a sleep period and a wake period for the subject. In some cases, a sleep-wake cycle may be referred to as a sleep-activity cycle or rhythm.

During a wake period, the subject may be awake, including, both alert and relaxed states of being awake. When a subject is alert, an EEG records beta waves, including the highest frequency and lowest amplitude waves. When a subject is relaxed, an EEG records alpha waves.

During a sleep period, the subject may be asleep, including, for example, one or more stages of sleep such as non-rapid eye movement sleep (NREM), and rapid eye movement sleep (REM) in which the subject may cycle through multiple times during a sleep period. In some cases, there may be one or more stages of NREM, including a light sleep or stage 1 (N1) sleep, deeper sleep or stage 2(N2 ), deep sleep or stage 3(N3 ).

N1 sleep is the lightest stage of sleep and an EEG records theta waves. N1 sleep may last 1 to 5 minutes, or approximately 5% of a sleep period.

N2 sleep is deeper than N1 and both heart rate and body temperature begin to drop. An EEG records sleep spindles and/or K-complexes. Sleep spindles are brief bursts of neural activity in the superior temporal gyri, anterior cingulate, insular cortices, and thalamus, to induce calcium influx into cortical pyramidal cells facilitating memory consolidation. K-complexes are long (e.g., one second) delta waves which also facilitates memory consolidation. N2 lasts around 25 minutes during the first sleep cycle, and gradually lengthen each successive cycle totaling approximately 45% of a sleep period.

N3 sleep is deeper than N2 and is the deepest non-REM sleep. An EEG records delta waves characterized by low frequencies and high amplitudes. N3 totals approximately 25% of a sleep period. During this stage, the body repairs and regrows tissue, builds bone and muscle, and strengthens and repairs the immune system. When a subject is awoken during N3, the subject may have moderately impaired mental performance for about 30 to 60 minutes. Sleepwalking, night terrors, and bedwetting occur during N3.

REM sleep is dreaming sleep. An EEG records beta waves, which are similar to during a wake period. Skeletal muscles are atonic, while eyes and diaphragm are active. Breathing is also erratic and irregular. REM lasts around 10 minutes during the first sleep cycle, and gradually lengthen each successive cycle to approximately 60 minutes totaling approximately 25% of a sleep period. Dreaming and nightmares occur during REM sleep, and oxygen use increases, as well as increased and variable pulse and blood pressure.

Sleep disorders, for example, sleep apnea, REM sleep disorder, narcolepsy, or somnambulism (e.g., sleepwalking), may disrupt or alter a sleep period, including shortening, lengthening, or skipping stages of the sleep cycle. This, in some cases, may result in changes to a subject's sleep-wake cycle as well as the subject's circadian rhythm. Similarly, other disorders or conditions may also affect a subject's sleep-wake cycle, such as depression, age, stress, traumatic brain injury, alcohol or drug consumption, and/or the like. For example, newborns and infants under a year old often have shorter sleep cycles and daytime naps, while children's sleep period may decrease to around 11 hours.

In some aspects, the subject's sleep-wake cycle may be associated with the subject's circadian rhythm. For example, a sleep period may be associated with a fourth period of the subject's circadian rhythm and a wake period may be associated with a second period of the subject's circadian rhythm.

The therapy scheduling component 116 is configured to determine a therapeutic schedule based on the subject's personalized circadian rhythm and the pharmacological data of the therapeutic(s) to be used to control a device, such as a device configured to administer a therapeutic. For example, a therapeutic schedule may include one or more control parameters (e.g., operational parameters), for controlling and causing the device to administer the therapeutic. In the depicted example, the therapy scheduling component 116 is configured to obtain pharmacological data of the therapeutic stored in a pharmacy library 110. The pharmacy library 110 includes pharmacological data associated with various therapeutics, for example, pharmacokinetics and pharmacodynamics of a therapeutic. Other pharmacological data may include indications which the therapeutic is intended to treat, a mechanism of action, dosage and administration information, contraindications, drug interactions, adverse effects, and/or clinical study data.

A therapeutic schedule may comprise a plan outlining a sequence or order of control of one or more devices, such as for controlling a device, such as for administration of a therapeutic. A therapeutic schedule may include control of one or more devices configured to provide one or more therapies, such as for a subject, including dosage information for each therapy. For example, the amount, frequency, route of administration, duration, and/or the like of each therapy for administration. Additionally, a therapeutic schedule may include a target time or time period for administration. Beneficially, a target time or time period may coincide with a point of the circadian rhythm to enhance efficacy and/or reduce adverse effects of the therapy.

In some aspects, the therapy scheduling component 116 is configured to determine a therapeutic schedule based on deterministic approach. For example, where a cortisol peak is 8:00, a dose of prednisone (a synthetic corticosteroid), is scheduled for 8:00 to coincide with the cortisol peak. As another example, a dose of a medication associated with drowsiness, such as an antihistamine, may be schedule for a third period of the circadian rhythm.

In some aspects, the therapy scheduling component 116 is configured to utilize one or more machine learning models 106 to determine a therapeutic schedule. For example, a machine learning model 106 may be configured to utilize the circadian rhythm and pharmacological data of the therapeutic(s) to be administered to determine a therapeutic schedule.

The infusion pump controlling component 118 is configured to control, or cause, an infusion pump 108, or other medical device to provide (e.g., administer) the therapeutic(s) according to the therapeutic schedule determined by the therapy scheduling component 116. In some aspects, the infusion pump controlling component 118 is configured to control a dialysis machine, such as a dialysis pump, to provide the therapeutic(s) according to the therapeutic schedule determined by the therapy scheduling component 116. For example, the infusion pump controlling component 118 may be configured to utilize one or more instructions in the control schedule to control operations of the infusion pump 108 to provide therapeutics.

Thereby, beneficially, the therapy may have improved efficacy and reduced adverse events due to timing with one or more circadian-dependent biological functions, especially in cases where the subject's circadian rhythm is altered or shifted, such as due to disease or treatment. Thus, improved treatment and survival outcomes, as well as improved patient quality of life may be achieved.

### Example Workflow for Personalized Chronotherapy

FIG. 2 depicts an example workflow 200 for controlling personalized chronotherapy, such as for a subject. For example, aspects of workflow 200 include determining a subject's personalized circadian rhythm and determining a therapeutic schedule based on the subject's personalized circadian rhythm and the pharmacology of the therapy. In some embodiments, aspects of workflow 200 may be performed by personalized chronotherapy system 100 in FIG. 1, for example, by personalized chronotherapy component 120.

Workflow 200 begins at step 212 with detecting sensor data captured by sensor 102 and/or smart device 104, for example, with sensor component 112 described with respect to FIG. 1. As described herein, sensor 102 and/or smart device 104 may be associated with a subject and be configured to sense one or more biomarkers of the subject. For example, sensor data may include cortisol, melatonin, GH, testosterone, insulin, glucose, TSH, T3, T4, epinephrine, sodium, potassium, chloride, lactose, urea, ammonia, short-chain fatty acids, creatinine, estrogen, activity of the subject, a HR, a heart rate reserve, a HRV, respiration rate, ECG data, temperature, impedance data, and/or the like associated with the subject. In some aspects, additional data may include a subject's medical history, one or more comorbidities, age, sex, current therapeutics, and/or the like. In some aspects, environmental data may include the environmental conditions associated with the subject, such as environmental temperature, environmental light conditions, and environmental noise conditions.

In some aspects, sensor data is detected over a period of time, for example, a 24-hour period, 3 days, a week, a month, etc. Thus, in certain aspects, sensor data may be correlated with a time within the period of time, e.g., sensor data may be timestamped.

In some aspects, at step 213, sensor data may be used to determine a sleep-wake cycle of the subject. For example, sensor data including activity data (e.g., using an accelerometer), breathe data, HR data, and/or the like may be used to determine whether the subject is asleep or awake. In addition, in some cases, environmental data such as environmental lighting conditions or environmental noise conditions may also be used to determine whether the subject is asleep or awake.

In some aspects, a sleep-wake cycle may be determined by determining a sleep period. A sleep period, in some aspects, may be determined based on a biomarker value of the sensor data obtained at step 212 satisfying a sleep threshold value for the biomarker. The time of the sensor data may be associated with the biomarker value, e.g., timestamped, and the time of the sensor data may be labeled as a sleep period. Example biomarkers for a sleep period may include one or more of a HR, a temperature, a respiration rate, an electrical activity of the brain, an activity or movement of the subject, and/or the like. For example, a respiration rate of the subject may satisfy a threshold for a sleep period where the respiration rate is below the threshold. The time which the subject's respiration rate was below the threshold may be labeled as a sleep period of the subject's sleep-wake cycle.

In some aspects, two or more biomarker values in the sensor data obtained at step 212 may be used to determine a sleep period. For example, where an HR and respiration rate for a time each satisfy respective thresholds for sleep period, the time may be labeled as a sleep period of the subject's sleep-wake cycle.

In some aspects, a sleep-wake cycle may be determined by determining a wake period. A wake period, in some aspects, may be determined based on a biomarker value of the sensor data obtained at step 212 satisfying a wake threshold value for the biomarker. The time of the sensor data may be associated with the biomarker value, e.g., timestamped, and the time of the sensor data may be labeled as a wake period. Example biomarkers for a wake period may include one or more of an HR, a temperature, a respiration rate, an electrical activity of the brain, an activity or movement of the subject, or the like.

In some aspects, two or more biomarker values in the sensor data obtained at step 212 may be used to determine a sleep period. For example, where an HR and respiration rate for a time each satisfy respective thresholds for sleep period, the time may be labeled as a sleep period of the subject's sleep-wake cycle.

In some aspects, a subject's sleep-wake cycle may be further based on additional data associated with the subject, for example, a subject's medical history, one or more comorbidities, age, sex, current therapeutics, and/or the like. For example, in some aspects, a sleep threshold may be based on additional data associated with the subject, such as the subject's age. In another example, a wake threshold may be based on additional data associated with the subject, such as the subject's age.

In some aspects, one or more machine learning models 204 may be used to determine a subject's sleep-wake cycle. The machine learning model 204 may be an example of the one or more machine learning models 106 described with respect to FIG. 1.

A machine learning model is a mathematical representation or algorithm which is used to recognize patterns, make predictions, or perform specific tasks, called “inferencing”. Generally, a model uses input data and its training to generate an output. A model may be characterized based on the type of learning used to generate and train the model. Example types of learning include supervised learning, unsupervised learning, and reinforcement learning.

Supervised learning trains the model based on a training dataset containing labeled data. Pairs of input and output data are used to train the model to adjust its parameters to minimize the difference (e.g., the loss) between its predictions based on the input, and the paired output. Some example supervised learning algorithms include nearest neighbor, naive Bayes, decision trees, linear regression, support vector machines (SVMs), and artificial neural networks (ANNs).

Unsupervised learning trains the model based on a training dataset without labeled data. The model is tasked to find patterns, relationships, and groupings between the input data without guidance.

Reinforcement learning trains the model based on feedback for actions taken by the model. An agent is the learner which decides the actions to take in the environment. Then, each action receives feedback in the form of a reward (positive) or penalty (negative). The agent learns to maximize rewarding actions and minimize penalized actions.

Regression models, such as linear regression and lasso or ridge regression may be used in some examples. Linear regression may be useful for making predictions based on time.

Tree-based models, such as random forest, decision trees, as well as boosted versions, such as gradient boosted tree models, may be useful for non-linear relationships and complex patterns.

Some neural network models, including ANNs, recurrent neural networks (RNNs), long short-term memory (LSTM), Graphical Neural Networks (GNNs), and/or the like may also be used. Neural networks may be used to learn from and make predictions for sequential data.

The machine learning model 204 may be used, in some aspects, to find patterns, including time-dependent patterns, between the sensor data detected at step 212, to determine the subject's sleep-wake cycle.

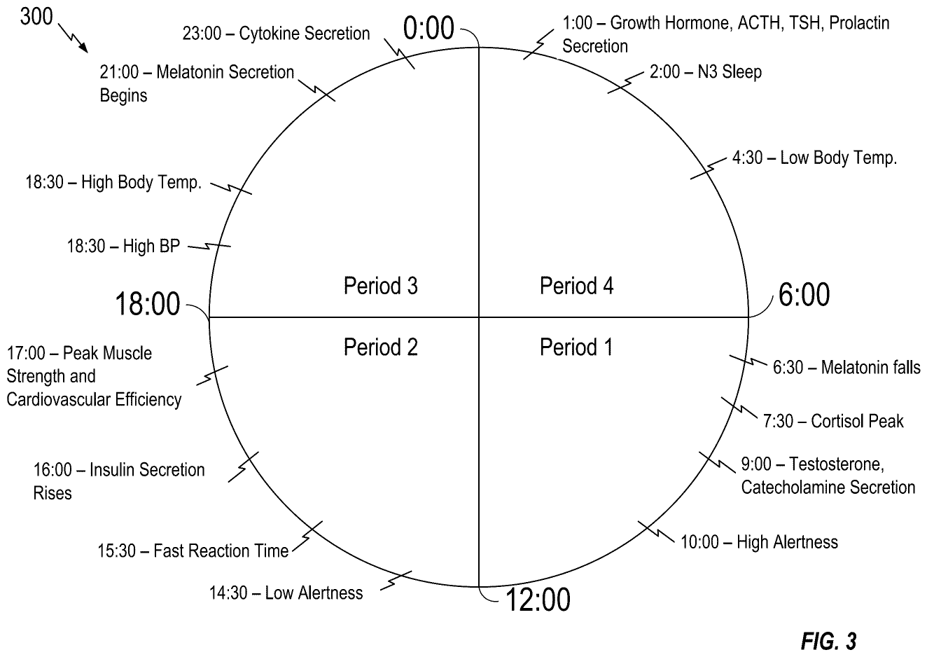

At step 214, sensor data, including, for example, cortisol and/or melatonin data may be used to determine a circadian rhythm, e.g., of a subject. In some aspects, the sensor data used at step 214 may include biomarker data. FIG. 3 depicts an example personalized circadian rhythm 300.

In some aspects, a circadian rhythm may be determined in a deterministic matter, for example, based on an biomarker satisfying a threshold, e.g., a maximum or minimum threshold, or by comparison to a reference value. For example, a cortisol value above a maximum threshold at 8:00 may be considered a morning peak.

In some aspects, a circadian rhythm may be determined based on identification of a peak or minimum value of a biomarker, and/or a trend of the biomarker. For example, an inflection point, e.g., a point in which a value of a biomarker transitions from increasing to decreasing or stabilizes (e.g., increasing to no change), may be categorized as a peak, while a transition from decreasing to increasing, or stabilizes (e.g., decreasing to no change) may be categorized as a minimum.

In some aspects, a trend (e.g., increasing, decreasing, no change) of a biomarker may be determined based on a comparing a value at a first time to a value at a second time. Where the value at the second time is higher than at the first time, the biomarker may be increasing. Where the value at the second time is lower than at the first time, the biomarker may be decreasing. No change may be determined where the value at the first time is equal, or approximately equal, to the value at the second time.

In some aspects, one or more values (e.g., peaks, minimums, trends, etc.) at the time of measurement may be correlated with a reference value and reference circadian rhythm. For example, a reference circadian rhythm includes a reference peak of cortisol around 8:00 (e.g., beginning of a day period). The reference circadian rhythm may also include a reference peak of melatonin around 22:00 (e.g., within a night period). Thus, a subject with a peak of cortisol around 7:00 and a peak of melatonin around 20:00 may be determined to have a circadian rhythm earlier than the reference circadian rhythm (e.g., shifted early by 2 hours).

In some cases, a subject's circadian rhythm may be determined to be approximately less than or greater than 24 hours.

In some aspects, a subject's circadian rhythm may be further based on additional data associated with the subject, for example, a subject's medical history, one or more comorbidities, age, sex, current therapeutics, and/or the like. For example, as described herein, some health conditions, therapeutics, and/or the like may alter a subject's circadian rhythm. Additionally, in some examples, some health therapeutics may alter a subject's circadian rhythm. As one example, a subject may administer a corticosteroid during a night period (e.g., 20:00) resulting in an increase in cortisol which is not associated with a day period, thus an alteration in the subject's circadian rhythm.

Further, in some examples, age, sex, previous health conditions, and the like, may also alter a circadian rhythm. In some examples, a reference circadian rhythm may be selected based on correlation between a characteristic of the subject and the reference circadian rhythm, for example, a first reference circadian rhythm may be associated with children (e.g., ages 6-12), and a second reference circadian rhythm may be associated with older adults (e.g., ages 60-75). A subject's age 68 may be compared to the second reference circadian rhythm.

In some aspects, a subject's circadian rhythm may be further based on environmental data associated with the subject, for example, environmental conditions. For example, environmental conditions may indicate or be associated with an activity of the subject, such as bright light or loud noises associated with an awake period. As another example, environmental conditions such as low or no light or quiet may be associated with a sleep period.

In some aspects, the circadian rhythm may be determined with one or more machine learning models, for example, machine learning model 206. The machine learning model 206 may be an example of the one or more machine learning models 106 described with respect to FIG. 1. In some aspects, the machine learning model 206 may be the same as the machine learning model 204, a different model, or a different type of model.

The machine learning model 206 may be used, in some aspects, to find patterns, including time-dependent patterns, between the sensor data detected at step 212, to determine the circadian rhythm.

As described herein, a personalized circadian rhythm may be characterized by timing of one or more events, for example, physiological events, activity events, and/or environmental events. FIG. 3 depicts a graphic depiction of a personalized circadian rhythm 300.

In personalized circadian rhythm 300, the features and activity of biomarkers, user activity, and/or the like, may be expected or predicted based on sensor data detected at step 212. Personalized circadian rhythm 300 is divided into the four periods, period 1 lasting from 6:00 to 12:00, may be an expected wake up time to mid-wake period. During period 1, the subject's melatonin level may fall (e.g., expected at 6:30), while cortisol peaks (e.g., expected at 7:30). Further, during period 1, the subject may be expected to experience increasing levels of other hormones such as testosterone and catecholamine. The subject may also be expected to experience peak or high alertness and mental acuity.

Period 2 may last from mid-wake period to beginning of a wind-down period. During period 2, the subject's alertness may drop (e.g., an afternoon dip), but experience increased reaction time, insulin secretion, and muscular strength and cardiovascular efficiency. Insulin secretion may also rise and digestion may proceed.

Period 3 may last from beginning of a wind-down period to when the subject sleeps. During period 3, the subject's blood pressure and body temperature may rise or peak. Towards the end of the period, melatonin secretion may begin in preparation for sleep. Further, immunological processes may increase with release of cytokines as the subject falls asleep. Cytokines may also be associated with regulation of a sleep, including sleep cycles during a sleep period.

Period 4 may last from when the subject sleeps to when the subject wakes. During period 4 the subject may sleep, with deepest sleep around 2:00. Hormones related to growth and metabolism may be secreted, including growth hormone, ACTH, TSH, and prolactin.

As described herein, the subject's circadian rhythm may shift, alter, or otherwise change. For example, as the subject ages the subject's sleep, e.g., period 4 may decrease with the subject waking earlier. As another example, health conditions may alter the subject's circadian rhythm, including, for example, cancer and other immunological disorders, cardiovascular system disorders, nervous system disorders, respiratory system disorders, digestive system disorders, endocrine and metabolic disorders, or musculoskeletal disorders.

Furthermore, in some cases, treatments, such as chemotherapy, may affect a subject's circadian rhythm. For example, chemotherapeutic agents such as anthracyclines (e.g., daunorubicin, doxorubicin, and epirubicin), aklylating agents (e.g., cyclophosphamide, ifosfamide, melphalan, and busulfan), platinum (e.g., ciplatin and oxaliplatin), antimetabolites (e.g., 5-fluorouracil, capecitabine, methotrexate, and gemcitabine), topoisomerase inhibitors (e.g., topotecan, irinotecan, etopside, and teniposide), mitotic inhibitors (e.g., paclitaxel, docetaxel, vinblastine, and vincristine), and molecular-targeted agents (e.g., transtuzumab), may affect a subject's circadian rhythm. Such therapeutics are often toxic and/or have severe adverse effects.

Returning to FIG. 2, at step 216, the subject's circadian rhythm is used to determine a therapeutic schedule for the subject. Additionally, in some aspects, pharmacological data associated with one or more therapeutics to be administered to the subject are also used. In the depicted example, pharmacological data for each therapeutic is stored in the pharmacy library 110. The pharmacy library 110 includes pharmacological data associated with various therapeutics, for example, pharmacokinetics and pharmacodynamics of therapeutics. Other pharmacological data may include indications which the therapeutic is intended to treat, a mechanism of action, dosage and administration information, contraindications, drug interactions, adverse effects, and/or clinical study data.

As described herein, the therapeutic schedule may comprise a plan outlining a sequence or order of therapies for administration, including, for example, the amount, frequency, route of administration, duration, and/or the like of each therapy for administration. Additionally, a therapeutic schedule may include a target time or time period for administration, for example, administer at a 14:00 dose time, or administer during a third period. Beneficially, a target time or time period may coincide with a point of the subject's circadian rhythm to enhance efficacy and/or reduce adverse effects of the therapy. Further, in some aspects, the infusion pump controlling component 118 may be configured to utilize one or more instructions in the therapeutic schedule (e.g., control schedule) to control operations of the infusion pump 108 to provide therapeutics.

For example, a therapeutic schedule may indicate to administer a corticosteroid at 7:00, which is associated with the subject's circadian rhythm peak of cortisol. As another example, an antidepressant may be administered at 20:00, which is associated with the subject's circadian rhythm increase in serotonin.

In another example, melatonin, in addition to regulation of sleep, may also provide a protective effect against chemotherapy drug-induced toxicity. Thus, in some cases, it may be beneficial to time administration of chemotherapy with the subject's melatonin secretion.

In some aspects, dosing information may also be set by the therapeutic schedule. Dosing parameters, such as operational parameters of an infusion pump system, including infusion rate and total infusion time may be associated with adverse consequences or risks. Thus, in some aspects, the therapeutic schedule may include dosing parameters such as infusion rate and total infusion time.

In some aspects, a therapeutic schedule is determined using a deterministic approach. For example, dosing indications such as mechanism of action, dosage timing and activity periods, adverse effects, circadian rhythm mechanisms affected, and/or the like, may be used to determine a therapeutic schedule.

For example, in some aspects, to determine the therapeutic schedule, a period of administration for the therapy may be determined based on the pharmacological data associated with the therapy. Pharmacological data associated with the therapy may include, for example, pharmacokinetic and/or pharmacodynamic information. For example, based on the metabolism of the therapy, the therapy is scheduled for administration with food. As another example, based on the therapeutic effects of the therapy, the therapy is scheduled for administration before sleep. One example of such a medication is an antihypertensive which may be more efficacious and help control blood pressure while the subject sleeps.

For example, chemotherapeutic agents may have improved efficacy when administered at times of best tolerability. Further, some biological functions may affect pharmacokinetics, such that dosing time may be timed to coincide with increased activity, and/or dosing adjusted where timing may indicate reduced activity. For example, processing of the therapy from entry of the therapy into the subject to clinical response may be affected by circadian-controlled biological functions. Specifically, in one example, short elimination half-life therapies may be timed to reach the target protein during their highest expression level. Additionally, diurnal oscillations in drug absorption, distribution, metabolism, and excretion, as well as daily variations in the sensitivity of molecular drug targets may resulting dosing-time changes, as well as the drug effectiveness and safety.

Next, the period of administration may be correlated with the period or time of the circadian rhythm, for example, a subject may consume food during a first period and a third period, and thereby the period of administration may be correlated with a first period or third period. As another example, the antihypertensive may be scheduled for administration just before sleep and the subject may sleep during the fourth period. Thus, the period of administration may be correlated with the fourth period.

In some aspects, a certain time or time period within a period of a circadian rhythm may be correlated, for example, the beginning of the fourth period, the end of the first period, and/or the like. In some cases, each period may be associated with a specific time, for example, the beginning of the fourth period may be 0:00, the end of the first period may be 12:00, and/or the like.

Then, the therapy may be scheduled for administration during the period of the circadian rhythm or at the time within the period of the circadian rhythm.

For example, pharmacological data for cancer patients treated with constant-rate intravenous infusion of 5-fluorouracil (5-FU) for 5 days may indicate that the maximum plasma concentration and the best-tolerated time to be at 4:00, and thus, the therapeutic schedule may schedule 5-FU infusion to start at 4:00.

As another example, pharmacological data for irinotecan may indicate fewer or less severe adverse effects when infused overnight, e.g., between 2:00 and 8:00, for 30 minutes, compared to conventional morning (e.g., period 1) infusion. Thus, the therapeutic schedule may schedule irinotecan infusion to start at 3:00. However, for a shift-worker, or otherwise altered circadian rhythm subject, the subject may sleep from 14:00 to 20:00, and thus, the irinotecan infusion may be scheduled for 18:00 to coincide with the subject's personal period 4.

Furthermore, in some aspects, a therapeutic schedule may also indicate other activities, such as food consumption, exercise, or the like, in conjunction with administration of a therapeutic. For example, for an antibiotic scheduled for administration at 12:00, the therapeutic schedule may also indication to administer food at 12:00 coinciding with the antibiotic administration to reduce adverse effects and/or increase absorption of the antibiotic.

In some aspects, the subject's therapeutic schedule may be determined with one or more machine learning models, for example, machine learning model 208. The machine learning model 208 may be an example of the one or more machine learning models 106 described with respect to FIG. 1. In some aspects, the machine learning model 208 may be the same as the machine learning model 204 and/or the machine learning model 206, a different model, or a different type of model.

The machine learning model 208 may be used, in some aspects, to find patterns, including time-dependent patterns, optimization, or scheduling, to determine the therapeutic schedule. For example, a physiologically-based PK-PD model may be used to, at least in part, to model the pharmacokinetics and pharmacodynamics of one or more therapies of the therapeutic schedule.

The control schedule may be generated using a machine learning model that receives a set of inputs, including one or more of a circadian rhythm (e.g., personalized circadian rhythm 300 in FIG. 3), patient information, therapeutic information, delivery mechanism (e.g., infusion pump, oral dose, syringe, gravity infusion, etc.), delivery device (e.g., type of infusion pump), or the like and provides as an output, a control schedule that can be used to control administration of the therapeutic. In examples, where a delivery device is used, the control schedule may be formatted as machine readable configuration information to adjust the delivery device in accordance with the control schedule.

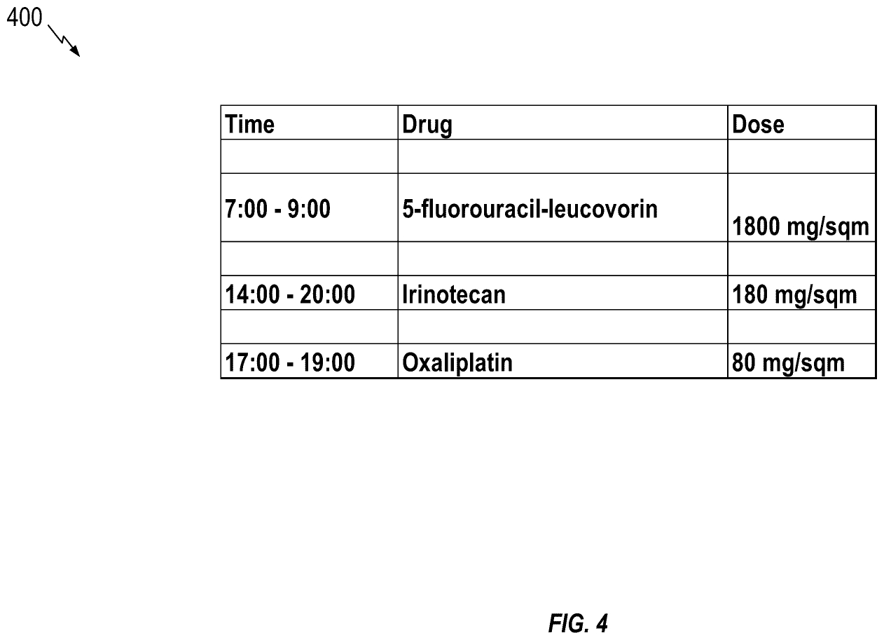

FIG. 4 depicts an example therapeutic schedule for administration of therapy to the subject. For example, in some aspects, the example therapeutic schedule may further include instructions for controlling a device configured to deliver a therapeutic (not depicted). In particular, FIG. 4 depicts an example therapeutic schedule 400 for administration of chemotherapy, in this example, 5-FU for treatment of colorectal, breast, or gastric cancer.

5-FU is often administered as a continuous infusion over several days, for example, 4 or 5 days. In some cases, multiple chemotherapeutic agents may be co-administered. For example, in an IFLO5 regimen, folinic acid and 5-FU (often together as fluorouracil-leucovorin), oxaliplatin, and irinotecan (a topoisomerase inhibitor) are administered in a cycle.

Over a 4 day cycle, fluorouracil-leucovorin is scheduled for administration at 1800 mg/m² intravenously using an infusion pump (e.g., infusion system 500 in FIG. 5) for a period of 2 hours starting at 7:00.

Oxaliplatin is scheduled for administration at 80 mg/m² intravenously using an infusion pump (e.g., infusion system 500 in FIG. 5) for a period of 2 hours starting at 17:00.

Irinotecan is schedule for administration at 180 mg/m² intravenously using an infusion pump (e.g., infusion system 500 in FIG. 5) for a period of 6 hours starting at 14:00.

In some aspects, dosing information may also be set by the therapeutic schedule. Dosing parameters, such as operational parameters of an infusion pump system, including infusion rate and total infusion time may be associated with adverse consequences or risks. Thus, in some aspects, the therapeutic schedule may include dosing parameters such as infusion rate and total infusion time.

Returning to FIG. 2, at step 218, the therapy may be administered in accordance with the therapeutic schedule. Additionally, or alternatively, at step 218, a device configured to deliver a therapeutic may be controlled based on a control schedule. For example, in some aspects, a control schedule may include instructions for controlling a device configured to deliver a therapeutic.

For example, in some aspects, where the therapy comprises an oral medication, the medication may be administered to the subject at the time or period specified in the therapeutic schedule for oral ingestion. In another example, where the therapy comprises a topical medication, the medication may be administered to the subject at the time or period specified in the therapeutic schedule for topical application. Where the therapy comprises an inhaled or intranasal medication, for example, the medication may be administer to the subject at the time or period specified in the therapeutic schedule for inhalation or intranasal spray.

In yet another example, where the therapy comprises a transdermal medication, the medication patch may be applied or worn by the subject for the time or period specified in the therapeutic schedule for transdermal administration. In some cases, a transdermal patch may be applied or worn for a period of time, or between two times, for example, during a first period, or between 6:00 and 18:00.

As another example, where the therapy comprises an injection, such as an intramuscular or subcutaneous injection, the therapy may be injected at the time or during the period specified in the therapeutic schedule.

Where the therapy comprises an intravenous medication, the therapy, for example, may be administered at the time or during the period specified in the therapeutic schedule. In some aspects, the therapeutic schedule may indicate a program for an infusion pump, including flow rate, duration, volume to be infused, bolus dose, infusion mode, and/or other operational parameters for an infusion pump to infuse the therapy in accordance with the therapeutic schedule. For example, a therapy may be scheduled to begin and/or end at a certain time, for a certain period, or the like. In one example, a therapy may be scheduled to be infused from 2:00 to 6:00. In another example, a therapy may be schedule to be infused for 4 hours during a fourth period.

In some cases, an intravenous medication may be scheduled for intermittent administration, for example, infuse a dose (e.g., a bolus dose) every 4 hours, infuse a dose at the beginning of every period, infuse a dose for 3 hours every third period, or the like.

In some aspects, the program for the infusion pump may be transmitted to the infusion pump controller, for example, infusion pump controller component 118 in FIG. 1, or a controller associated with infusion system 500 in FIG. 5.

Thus, beneficially, in some aspects, administration of a therapy may be administered and/or controlled in accordance with the subject's circadian rhythm.

In some aspects, one or more steps of workflow 200 may be repeated, for example, steps 212-214 may be repeated to account and adjust for changes in a subject's circadian rhythm. For example, a subject with a chronic or sever disorder, such as cancer, may progress and their circadian rhythm may further change. Alternatively, as a subject improves, e.g., in remission, their circadian rhythm may also change. Further, steps 216-218 may also be repeated based on changes in the subject's circadian rhythm.

In some aspects, workflow 200 may repeat steps 216-218 when a subject's therapy changes, for example, a therapy is added or removed, a dosage is updated, or the like.

For example, in some aspects, workflow 200 may be repeated between treatment cycles, such as between cycles of chemotherapy. In some aspects, workflow 200 may be repeated on a regular schedule, for example, monthly, annually, bi-annually, or the like.

Thereby, beneficially, the therapy may have improved efficacy and reduced adverse events due to timing with one or more circadian-dependent biological functions, especially in cases where the subject's circadian rhythm is altered or shifted, such as due to disease or treatment. Thus, improved treatment and survival outcomes, as well as improved patient quality of life may be achieved.

Note that workflow 200 is just one example, and other flows including fewer, additional, or alternative steps, consistent with this disclosure, are possible.

### Example Infusion System

FIG. 5 depicts an example infusion system 500, which may implement methods described herein for personalized chronotherapy. The infusion system 500 may be coupled to a patient 550 to administer a substance to the patient 550 through infusion. The infusion system 500 may include one or more pumps, for example, pump 502, pump 504, pump 506, and pump 508. Although a large volume pump is illustrated, other types of pumps may be implemented, such as a peristaltic pump, a small volume pump, a syringe pump, an anesthesia delivery pump, an ambulatory infusion pump, or a patient-controlled analgesic. A pump may be an infusion device configured to deliver a substance, (e.g., fluid, nutrients, drug, therapy, etc.) to a patient's circulatory system, or epidural space, for example, via an intravenous infusion, subcutaneous infusion, arterial infusion, epidural infusion, etc., or to a patient's digestive system, for example, via a nasogastric tube (NG), a percutaneous endoscopic gastrostomy tube (PEG), nasojejunal tube (NJ), etc. Each of the pumps 502, 504, 506, or 508 may comprise a standard infusion pumping unit, patient-controlled analgesia (PCA) pump, syringe pump, pulse oximeter, invasive or non-invasive blood pressure monitor, electrocardiograph, bar code reader, printer, temperature monitor, RF telemetry link, fluid warmer/IV pump, or high rate IV pump (2000+ ml/hr).

Each of the pumps 502, 504, 506, or 508 may be fluidly connected with an upstream fluid line 512, fluid line 514, fluid line 516, and fluid line 518, respectively. Further, each of pump 502, pump 504, pump 506, and pump 508, may be fluidly connected with a downstream fluid line 522, fluid line 524, fluid line 526, and fluid line 528, respectively. The fluid lines may be any type of fluid conduit, such as tubing, through which fluid can flow.

Each of fluid supply 532, fluid supply 534, fluid supply 536, and fluid supply 538, may be a reservoir, for example, as bottles shown, inverted and suspended above the pumps. Fluid supplies may also take the form of bags, syringes, or other types of containers. The medical device system 500 may be mounted on a roller stand or intravenous pole 540.

Infusion system 500 may further comprise check valves, drip chambers, valved ports, connectors, and other devices configured to administer a substance.

Each of the pumps 502, 504, 506, or 508 of infusion system 500 may be coupled to an infusion pump controller 560. The infusion pump controller 560 may be a patient care device (PCD) or patient care unit (PCU). The infusion pump controller 560 is configured to control operations of the pumps 502, 504, 506, or 508, for example, to control infusions to patient 550. The infusion pump controller 560 may be further configured to the control of fluid delivery to the patient and the monitoring of the fluid path for occlusion or air-in-line obstructions.

The infusion pump controller 560 may further include a graphical user interface (GUI), for example, an information display such as a liquid crystal display, and/or a touch-screen. The user interface of the infusion pump controller 560 may be used during set up and/or operating procedures to facilitate control of the infusion system 500. For example, one or more of entry, editing, or display of operational parameters of the pumps 502, 504, 506, or 508. The infusion pump controller 560 may further include one or more softkeys and/or hardkeys to facilitate data entry and commands. In some aspects, a user interface may display the operational parameters of the pumps 502, 504, 506, or 508, for example, the infusion rate at which the pump is operating. The user interface may additionally or alternatively, be used to display informational, advisory, alarm, or malfunction messages associated with the pumps 502, 504, 506, or 508.

The infusion pump controller 560 may further be configured to provide power to the infusion system 500 and interface between aspects of the infusion system 500 and external devices or networks, for example, patient monitoring networks, instructional systems (e.g., an electronic medical record system), pharmacy systems, or nurse call systems, or as an interface to external equipment such as barcode readers to provide a means of inputting drug and/or patient information from medication or patient records. In some aspects, the infusion pump controller 560 is configured to provide physical attachment of the infusion system 500 to structures, such as intravenous pole 540, bedrail(s), or the like.

### Example Syringe Pump

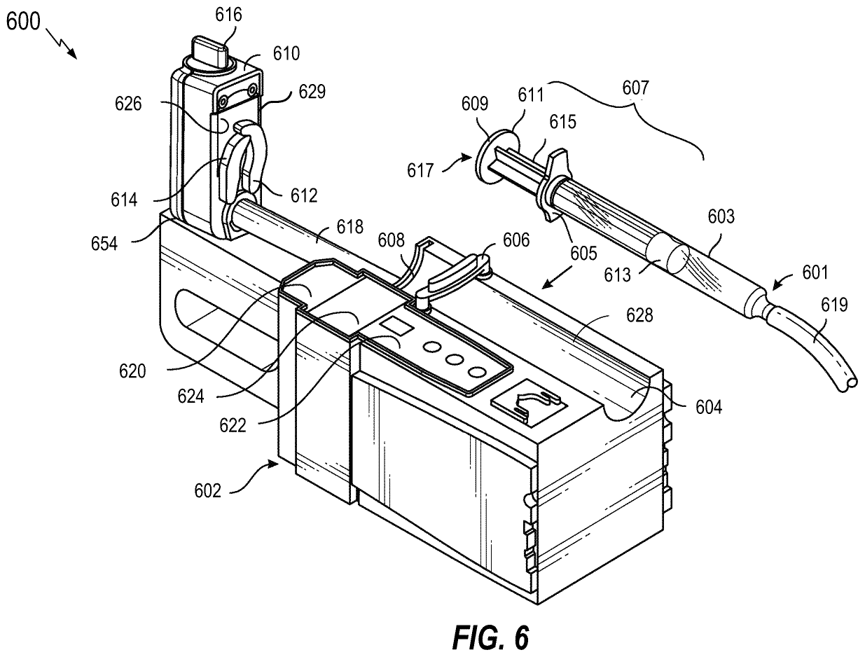

FIG. 6 depicts an example syringe pump 602. Syringe pump 602 includes a graphical user interface (GUI) 624, for example, an information display such as a liquid crystal display, and/or a touch-screen, for displaying operational information, including the selected syringe type and the configured infusion (e.g., in terms of rate of infusion, volume infused, length of time of infusion, etc.). A user may operate syringe pump 602 through one or more buttons 622 in this example. Note that other examples may include other arrangements, such as touch-screen interfaces, or interfaces operable by remote device, such as by an application on a computing device.

Syringe 601 is shown next to syringe pump 602, rather than loaded in the pump, for clarity of illustration. Syringe pump 602 includes cradle 604 in which a barrel 603 of syringe 601 rests when mounted in the syringe pump 602. Sensor 628 detects volume markings along a barrel of syringe 601. Cradle 604 has a clamp 606 to securely hold the barrel 603 in a fixed position in cradle 604 to resist axial and lateral movement. Clamp 606 may pivot between an open position to permit loading or removal of syringe 601 and a closed position over cradle 604. Barrel clamp 606 may measure an outside diameter of the barrel 603 of syringe 601. Barrel flange 605 of syringe 601 resides in a barrel flange groove 608 in syringe pump 602 to immobilize barrel 603 from axial movement during movement of plunger 607 within barrel 603 of syringe 601. Barrel flange groove may measure an outside diameter and a thickness of the barrel flange 605 of syringe 601.

Plunger 607 can include push-button 609 having an inner side 611 and being interconnected with stopper 613 of plunger 607 by piston 615. When mounted in syringe pump 602, push-button 609 can be held by drive head 610 with a plunger retainer comprising a pair of pivotally mounted claws, first retainer claw 612 and second retainer claw 614, shown in the closed position in FIG. 6. The retainer claws 612 and 614 can curve inwardly toward each other to grasp push-button 609 mounted in syringe pump 602.

A rotation knob 616 can be used to control the positions of the first and second retainer claws 612 and 614 to allow removal and insertion of the push-button 609 and to release the split-nut from the driveshaft to permit axial positioning of the drive head 610. Syringes can be provided for use with a syringe pump with different quantities of fluid, and the plunger can be located at different positions in relation to the barrel.

The drive head 610 can allow manual adjustment to accommodate syringes with different beginning plunger positions. A syringe inserted in the cradle 604 can align with the drive head 610 within a particular axial range. The points where the axial center lines of the syringe intersect the driver can change according to the size of the syringe but only in one direction along the drive head 610. A guide device 618 can extend from the drive head 610 to a point within a body of the syringe pump 602.

Syringe pump 602 can include a control panel 620 providing multiple buttons 622 for control of the syringe pump 602 as well as GUI 624 used to present pump-specific information to the operator. The buttons 622 can allow the operator to program the syringe pump 602 for the flow rate, the volume to be infused, and other pump parameters. GUI 624 can present the programmed flow rate, the amount of fluid remaining to be infused, as well as alarms and other information.

The drive head 610 can include a contact plate 626 that has a pushing surface that contacts the outer side 617 of the push-button 609 as the drive head 610 moves forward toward the barrel 603, pushing the plunger 607 into the barrel 603 of the syringe to expel the syringe contents through a fluid administration set tubing 619 to a patient. A drive mechanism 654 is configured to drive the drive head 610 forward. The drive mechanism 654 may include, a drivetrain, for example, gears, axles, shafts, chains, hydraulics, and/or any other components for translating rotational motion of a motor into linear motion of the drive head 610. The drivetrain may include linear actuating components, such as a linear stepper motor, a brushed DC electric motor, a brushless DC electric motor, a servo motor, an AC motor, or any other type of motor.

When the contact plate 626 exerts force against the push-button 609, the force may be detected by force sensor 629 and transmitted to a processor for monitoring. In the case of friction between the stopper 613 and the barrel 603, the force exerted can increase and can be detected by force sensor 629.

Syringe pump 602 is configured with known syringe types containing information such as syringe inner diameter and stroke of the stopper 613. Syringe pump 602 determines the position of stopper 613 based on the movement of drive head 610 based on the syringe type and stored characteristics of the syringe type. Then, syringe pump 602 calculates the volume infused, time elapsed, volume remaining, and time remaining. As the drive head 610 continues to move, a flow rate is determined based on the characteristics of the syringe and the velocity of the drive head 610.

One or more sensors, such as sensor 628, barrel clamp 606, groove 608, claws 612 and 614, and force sensor 629, may measure various characteristics of syringe 601, to obtain a measured value for the characteristic, such as barrel length, barrel outside diameter, flange shape, flange size, plunger shape, plunger size (e.g., diameter), plunger tab thickness, volume per plunger distance moved, driver head height, etc. These sensors may include one or more of an optical sensor, a light source, a pressure sensor, a position sensor (e.g., magnetic linear position sensor indicating the plunger head), force sensor, and the like.

### Example Personalized Chronotherapy Method

FIG. 7 shows an example personalized chronotherapy method 700 implemented by or under the direction of an infusion pump controller, such as part of infusion system 500 in FIG. 5.

Method 700 begins at step 702 with obtaining sensor data associated with one or more sensors, for example, as described with respect to step 212 of FIG. 2.

Method 700 then proceeds to step 704 with determining a circadian rhythm based on the sensor data, for example, as described with respect to step 214 of FIG. 2.

Method 700 then proceeds to step 706 with determining a control schedule, for a device configured to deliver a therapeutic, based on the circadian rhythm and pharmacological data associated with the therapeutic, for example, as described with respect to step 216 of FIG. 2.

Method 700 then proceeds to step 708 with controlling the device in accordance with the control schedule, for example, as described with respect to step 218 of FIG. 2.

In some aspects, the sensor data comprises a cortisol level of a subject.

In some aspects, the sensor data comprises a melatonin level of a subject.

In some aspects, the sensor data is obtained over at least a 24-hour time period.

In some aspects, step 704 includes processing the sensor data with a first machine learning model trained to determine the circadian rhythm.

In some aspects, step 704 includes: identifying a maximum level of a biomarker of the sensor data; associating the maximum level of the biomarker with a time of the sensor data; and labeling the time of the sensor data as a period of the circadian rhythm based on an attribute of the biomarker.

In some aspects, step 706 includes processing the circadian rhythm and the pharmacological data with a second machine learning model trained to generate the control schedule.

In some aspects, step 706 includes: determining a period of administration for the therapeutic based on the pharmacological data; correlating the period of administration with a period of the circadian rhythm; and scheduling the device to provide the therapeutic within the period of the circadian rhythm.

In some aspects, the pharmacological data comprises pharmacokinetic data and pharmacodynamic data of the therapeutic.

In some aspects, method 700 further includes determining a sleep-wake cycle based on the sensor data, wherein the circadian rhythm is further determined based on the sleep-wake cycle, for example, as described with respect to step 213 of FIG. 2.

In some aspects, determining the sleep-wake cycle comprises processing the sensor data with a third machine learning model trained to determine the sleep-wake cycle.

In some aspects, determining the sleep-wake cycle comprises: determining a biomarker value of the sensor data satisfies a sleep threshold value for a biomarker; associating the biomarker value with a time of the sensor data; and labeling the time of the sensor data as a sleep period of the circadian rhythm.

In some aspects, determining the sleep-wake cycle comprises: determining a biomarker value of the sensor data satisfies a wake threshold value for a biomarker; associating the biomarker value with a time of the sensor data; and labeling the time of the sensor data as a wake period of the circadian rhythm.

In some aspects, the device comprises an infusion pump system.

In some aspects, the device comprises a dialysis system. A dialysis system may comprise a dialysis pump configured to pump blood, and a dialyzer configured to filter blood.

In some aspects, the one or more sensors are associated with a subject; the circadian rhythm is determined for the subject; and the device is associated with the subject.

In some aspects, the device comprises an infusion pump.

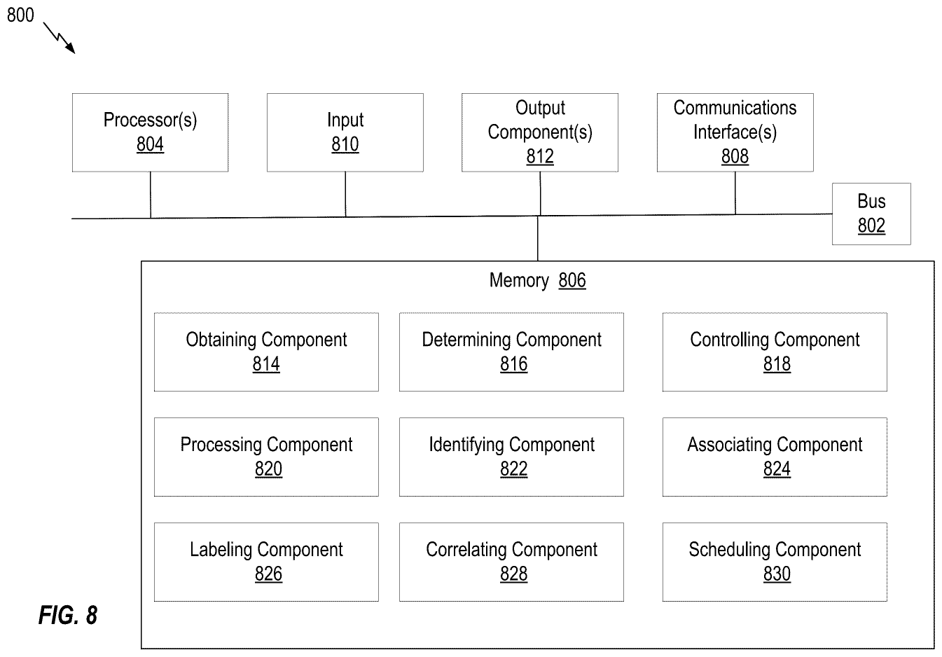

In one aspect, method 700, or any aspect related to it, may be performed by an apparatus, such as computing device 800 of FIG. 8, which includes various components operable, configured, or adapted to perform the method 700. Computing device 800 is described below in further detail.

Note that FIG. 7 is just one example of a method, and other methods including fewer, additional, or alternative steps are possible consistent with this disclosure.

### Example Computing Device

FIG. 8 depicts an example computing device 800, such as a medical device, that implements various features and processes described herein, such as infusion system 500 in FIG. 5, syringe pump 602, and/or a personalized chronotherapy method such as shown in FIG. 7. For example, the computing device 800 may perform one or more steps of any of flow 200 or method 700. The computing device 800 may include one or more processors 804, one or more memories 806, one or more input components 810, one or more output components 812, and one or more communication interfaces 808. Each of these components may be coupled by a bus 802.

Computing device 800 may perform these processes based on one or more processors 804 executing software instructions stored by a computer-readable medium, such as one or more memories 806. In certain embodiments, one or more processors 804 may be programmed/designed/configured to perform these processes. A computer-readable medium (e.g., a non-transitory computer-readable medium) is defined herein as a non-transitory memory device. A memory device includes memory space located inside of a single physical storage device or memory space spread across multiple physical storage devices. Software instructions may be read into one or more memories from another computer-readable medium or from another device via communication interface 808. When executed, software instructions stored in one or more memories may cause one or more processors 804 to perform one or more processes described herein.

A memory 806 may include data storage or one or more data structures (e.g., a database, etc.). Computing device 800 may be capable of receiving information from, storing information in, communicating information to, or searching information stored in the data storage or one or more data structures in one or more memories 806.

A memory 806 may include random access memory (RAM), read only memory (ROM), and/or other types of dynamic or static storage devices (e.g., flash memory, magnetic memory, optical memory, etc.), that stores information and/or instructions for use by one or more processors 804. For example, a memory 806 may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks.

In some aspects, memory 806 may include obtaining component 814, determining component 816, controlling component 818, processing component 820, identifying component 822, associating component 824, labeling component 826, correlating component 828, and scheduling component 830. In some aspects, one or more memories 806 may include obtaining component 814 configured for obtaining sensor data associated with one or more sensors. In some aspects, one or more memories 806 may include determining component 816 configured for determining a circadian rhythm based on the sensor data and determining a control schedule, for a device configured to deliver a therapeutic, based on the circadian rhythm and pharmacological data associated with the therapeutic. In some aspects, one or more memories 806 may include controlling component 818 configured for controlling the device in accordance with the control schedule. In some aspects, one or more memories 806 may include sensor data used by one or more of components 814-830.

One or more processors 804 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.), that may be programmed to perform a function, such as described herein.

One or more input components 810 may include a component that permits computing device 600 to receive information, such as via user input (e.g., a touch-screen display, a keyboard, a keypad, a mouse, a button, a switch, a microphone, etc.). Further, one or more input components 810 may include a sensor for sensing information (e.g., a global positioning system (GPS) component, an accelerometer, a gyroscope, an actuator, etc.).

One or more output components 812 may include a component that provides output information from computing device 800 (e.g., a display, a speaker, one or more light-emitting diodes (LEDs), etc.).

Communication interface 808 may include a transceiver-like component (e.g., a transceiver, a separate receiver and transmitter, etc.) that enables computing device 800 to communicate with other devices, such as via a wired connection, a wireless connection, or a combination of wired and wireless connections. Communication interface 808 may permit computing device 800 to receive information from another device and/or provide information to another device. For example, communication interface 808 may include an Ethernet interface, an optical interface, a coaxial interface, an infrared interface, a radio frequency (RF) interface, a universal serial bus (USB) interface, a Wi-Fi interface, a cellular network interface, and/or the like.

### Example Clauses

Implementation examples are described in the following numbered clauses:
Clause 1: A method, comprising: obtaining sensor data associated with one or more sensors; determining a circadian rhythm based on the sensor data; determining a control schedule, for a device configured to deliver a therapeutic, based on the circadian rhythm and pharmacological data associated with the therapeutic; and controlling the device in accordance with the control schedule.
Clause 2: The method of Clause 1, wherein the sensor data comprises a cortisol level of a subject.
Clause 3: The method of any one of Clauses 1-2, wherein the sensor data comprises a melatonin level of a subject.
Clause 4: The method of any one of Clauses 1-3, wherein the sensor data is obtained over at least a 24-hour time period.
Clause 5: The method of any one of Clauses 1-4, wherein determining the circadian rhythm based on the sensor data comprises processing the sensor data with a first machine learning model trained to determine the circadian rhythm.
Clause 6: The method of any one of Clauses 1-5, wherein determining the circadian rhythm based on the sensor data comprises: identifying a maximum level of a biomarker of the sensor data; associating the maximum level of the biomarker with a time of the sensor data; and labeling the time of the sensor data as a period of the circadian rhythm based on an attribute of the biomarker.
Clause 7: The method of any one of Clauses 1-6, wherein determining the control schedule comprises processing the circadian rhythm and the pharmacological data with a second machine learning model trained to generate the control schedule.
Clause 8: The method of any one of Clauses 1-7, wherein determining the control schedule comprises: determining a period of administration for the therapeutic based on the pharmacological data; correlating the period of administration with a period of the circadian rhythm; and scheduling the device to provide the therapeutic within the period of the circadian rhythm.
Clause 9: The method of any one of Clauses 1-8, wherein the pharmacological data comprises pharmacokinetic data and pharmacodynamic data of the therapeutic.
Clause 10: The method of any one of Clauses 1-9, further comprising determining a sleep-wake cycle based on the sensor data, wherein the circadian rhythm is further determined based on the sleep-wake cycle.
Clause 11: The method of Clause 10, wherein determining the sleep-wake cycle comprises processing the sensor data with a third machine learning model trained to determine the sleep-wake cycle.
Clause 12: The method of Clause 10, wherein determining the sleep-wake cycle comprises: determining a biomarker value of the sensor data satisfies a sleep threshold value for a biomarker; associating the biomarker value with a time of the sensor data; and labeling the time of the sensor data as a sleep period of the circadian rhythm.
Clause 13: The method of Clause 10, wherein determining the sleep-wake cycle comprises: determining a biomarker value of the sensor data satisfies a wake threshold value for a biomarker; associating the biomarker value with a time of the sensor data; and labeling the time of the sensor data as a wake period of the circadian rhythm.
Clause 14: The method of any one of Clauses 1-13, wherein the device comprises an infusion pump system.
Clause 15: The method of any one of Clauses 1-14, wherein the device comprises a dialysis system.
Clause 16: The method of any one of Clauses 1-15, wherein: the one or more sensors are associated with a subject; the circadian rhythm is determined for the subject; and the device is associated with the subject.
Clause 17: The method of any one of Clauses 1-16, wherein the device comprises an infusion pump.
Clause 18: The method of any one of Clause 1-16, wherein the device comprises an ambulatory infusion pump.
Clause 19: The method of any one of Clause 1-16, wherein the device comprises a syringe pump.
Clause 20: An infusion pump controller comprising: one or more memories; and one or more processors, coupled to the one or more memories, configured to perform a method in accordance with any one of Clauses 1-19.
Clause 21: A processing system, comprising: memory comprising computer-executable instructions; and one or more processors configured to execute the computer-executable instructions and cause the processing system to perform a method in accordance with any one of Clauses 1-19.
Clause 22: A processing system, comprising means for performing a method in accordance with any one of Clauses 1-19.
Clause 23: A non-transitory computer-readable medium storing program code for causing a processing system to perform the steps of any one of Clauses 1-19.
Clause 24: A computer program product embodied on a computer-readable storage medium comprising code for performing a method in accordance with any one of Clauses 1-19.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). Reference to an element in the singular is not intended to mean only one unless specifically so stated, but rather “one or more.” For example, reference to an element (e.g., “a processor,” “a memory,” etc.), unless otherwise specifically stated, should be understood to refer to one or more elements (e.g., “one or more processors,” “one or more memories,” etc.). The terms “set” and “group” are intended to include one or more elements, and may be used interchangeably with “one or more.” Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions. Unless specifically stated otherwise, the term “some” refers to one or more.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. An infusion pump controller comprising:
one or more memories; and
one or more processors, coupled to the one or more memories, configured to:
obtain sensor data associated with one or more sensors for a patient;
determine a circadian rhythm based on the sensor data;
determine a control schedule for an infusion pump configured to deliver a therapeutic to the patient, based on the circadian rhythm and pharmacological data associated with the therapeutic; and
cause the infusion pump to be configured for the patient in accordance with the control schedule.

2. The infusion pump controller of claim 1, wherein the sensor data comprises a cortisol level of the patient.

3. The infusion pump controller of claim 1, wherein the sensor data comprises a melatonin level of the patient.

4. The infusion pump controller of claim 1, wherein the sensor data is obtained over at least a 24-hour time period.

5. The infusion pump controller of claim 1, wherein, to determine the circadian rhythm based on the sensor data, the one or more processors are configured to process the sensor data with a first machine learning model trained to determine the circadian rhythm.

6. The infusion pump controller of claim 1, wherein, to determine the circadian rhythm based on the sensor data, the one or more processors are configured to:
identify a maximum level of a biomarker of the sensor data;
associate the maximum level of the biomarker with a time of the sensor data; and
label the time of the sensor data as a period of the circadian rhythm based on an attribute of the biomarker.

7. The infusion pump controller of claim 1, wherein, to determine the control schedule, the one or more processors are configured to process the circadian rhythm and the pharmacological data with a second machine learning model trained to generate the control schedule.

8. The infusion pump controller of claim 1, wherein, to determine the control schedule, the one or more processors are configured to:
determine a period of administration for the therapeutic based on the pharmacological data;
correlate the period of administration with a period of the circadian rhythm; and
schedule the infusion pump to provide the therapeutic within the period of the circadian rhythm.

9. The infusion pump controller of claim 1, wherein the pharmacological data comprises pharmacokinetic data and pharmacodynamic data of the therapeutic.

10. The infusion pump controller of claim 1, wherein the one or more processors are further configured to:
determine a sleep-wake cycle based on the sensor data, wherein the circadian rhythm is further determined based on the sleep-wake cycle.

11. The infusion pump controller of claim 10, wherein, to determine the sleep-wake cycle, the one or more processors are configured to process the sensor data with a third machine learning model trained to determine the sleep-wake cycle.

12. The infusion pump controller of claim 10, wherein, to determine the sleep-wake cycle, the one or more processors are configured to:
determine a biomarker value of the sensor data satisfies a sleep threshold value for a biomarker;
associate the biomarker value with a time of the sensor data; and
label the time of the sensor data as a sleep period of the circadian rhythm.

13. The infusion pump controller of claim 10, wherein, to determine the sleep-wake cycle, the one or more processors are configured to:
determine a biomarker value of the sensor data satisfies a wake threshold value for a biomarker;
associate the biomarker value with a time of the sensor data; and
label the time of the sensor data as a wake period of the circadian rhythm.

14. The infusion pump controller of claim 1, wherein the infusion pump comprises an ambulatory infusion pump.

15. The infusion pump controller of claim 1, wherein the infusion pump comprises a dialysis pump.

16. The infusion pump controller of claim 1, wherein the infusion pump comprises a syringe pump.

17. A method, comprising:
obtaining sensor data associated with one or more sensors;
determining a circadian rhythm based on the sensor data;
determining a control schedule, for a device configured to deliver a therapeutic, based on the circadian rhythm and pharmacological data associated with the therapeutic; and
controlling the device in accordance with the control schedule.

18. The method of claim 17, wherein determining the circadian rhythm based on the sensor data comprises processing the sensor data with a first machine learning model trained to determine the circadian rhythm.

19. The method of claim 17, wherein determining the control schedule comprises processing the circadian rhythm and the pharmacological data with a second machine learning model trained to generate the control schedule.

20. A non-transitory computer-readable medium storing program code for causing a processing system to perform the steps of:
obtaining sensor data associated with one or more sensors;
determining a circadian rhythm based on the sensor data;
determining a control schedule, for a device configured to deliver a therapeutic, based on the circadian rhythm and pharmacological data associated with the therapeutic; and
controlling the device in accordance with the control schedule.
